(19) United States
(12) Patent Application Publication
(10) Pub. No.: US 2026/0167653 A1
(43) Pub. Date: Jun. 18, 2026

(54) PICOLYL LIGANDS AND THEIR USE IN ALKOXYCARBONYLATION

(71) Applicant: Evonik Oxeno GmbH & Co. KG, Marl (DE)

(72) Inventors: Stefan Müller, Recklinghausen (DE); Peter Kucmierczyk, Herne (DE); Robert Franke, Marl (DE); Anna Chiara Sale, Düsseldorf (DE); Dirk Fridag, Haltern am See (DE); Ana Markovic, Haltern am See (DE)

(73) Assignee: Evonik Oxeno GmbH & Co. KG, Marl (DE)

(21) Appl. No.: 19/125,212

(22) PCT Filed: Oct. 27, 2023
(86) PCT No.: PCT/EP2023/080099

§ 371 Date: Apr. 28, 2025

(30) Foreign Application Priority Data
Nov. 4, 2022 (EP) 22205590.7

(51) Int. Cl. C07F 9/58; C07C 67/38

(52) U.S. Cl. CPC C07F 9/58; C07C 67/38

(57) ABSTRACT

The invention relates to picolyl ligands and their use in alkoxycarbonylation.

The invention relates to picolyl ligands and use thereof in alkoxycarbonylation.

The alkoxycarbonylation of ethylenically unsaturated compounds is a process of increasing significance. An alkoxycarbonylation is understood to mean the reaction of ethylenically unsaturated compounds (olefins) with carbon monoxide and alcohols in the presence of a metal-ligand complex to give the corresponding esters. Typically, the metal used is palladium. The following scheme shows the general reaction equation for an alkoxycarbonylation:

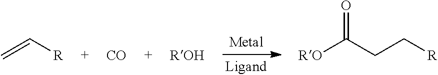

The technical object of the invention is to provide a novel ligand/process with which the conversion may be increased.

The object is achieved by a compound according to Claim 1.

### Compound of Formula (I):

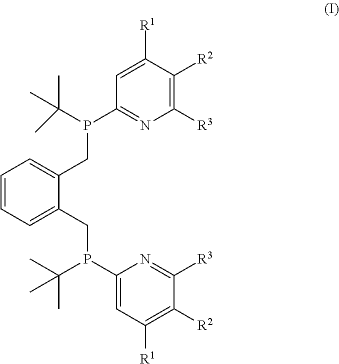

where one of the three radicals R¹, R², R³ is —(C₁-C₄)-alkyl, and the other two radicals are —H.

In one embodiment, one of the three radicals R¹, R², R³ is —CH₃, and the other two radicals are —H.

In one embodiment, the compound has the structure (1):

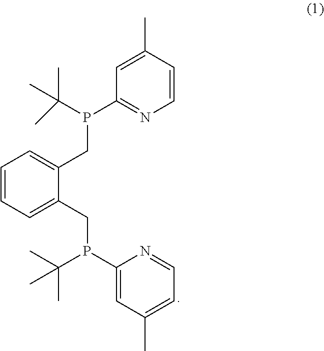 [SMILES: Cc1ccnc(P(Cc2ccccc2CP(c2cc(C)ccn2)C(C)(C)C)C(C)(C)C)c1]

In one embodiment, the compound has the structure (2):

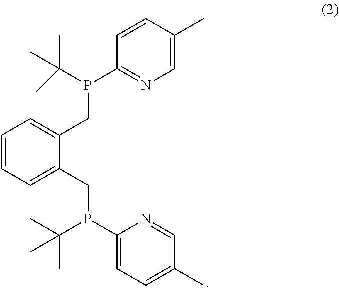 [SMILES: Cc1ccc(P(Cc2ccccc2CP(c2ccc(C)cn2)C(C)(C)C)C(C)(C)C)nc1]

In one embodiment, the compound has the structure (3):

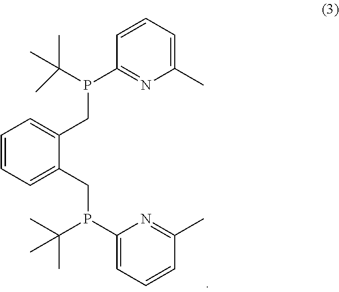 [SMILES: Cc1cccc(P(Cc2ccccc2CP(c2cccc(C)n2)C(C)(C)C)C(C)(C)C)n1]

In addition to the compounds per se, a process in which the compounds are used is also claimed.

Process comprising the process steps of:
a) initially charging an ethylenically unsaturated compound;
b) adding a compound of formula (I) described above;
c) adding a Pd compound;
d) adding a co-catalyst selected from: aluminium triflate, H₂SO₄, MSA, pTSA, TFA;
e) adding an alcohol;
f) feeding in CO;
g) heating the reaction mixture from a) to f), with conversion of the ethylenically unsaturated compound to an ester.

It is possible here to add the substances in any order. Typically, however, CO is added after the co-reactants have been initially charged in steps a) to e). In addition, CO can also be fed in in two or more steps, in such a way that, for example, a portion of the CO is first fed in, then the mixture is heated, and then a further portion of CO is fed in.

In one variant of the process, the Pd compound is selected from: palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl) dichloride.

In one variant of the process, the Pd compound is selected from: Pd(dba)₂, Pd(acac)₂ or Pd(OAc)₂.

In one variant of the process, the Pd compound is Pd(acac)₂.

In one variant of the process, the alcohol in process step e) is selected from: methanol, ethanol, 1-propanol, 1-butanol, 1-pentanol, 1-hexanol, 2-propanol, tert-butanol, 3-pentanol, cyclohexanol, phenol, or mixtures thereof.

In one variant of the process, the alcohol in process step e) is methanol.

In one variant of the process, the alcohol in process step e) is used in excess.

In one variant of the process, the alcohol in process step e) is used simultaneously as solvent.

In one variant of the process, CO is fed in in process step f) at a CO partial pressure in the range of 1 to 5 MPa (10 to 50 bar).

In one variant of the process, CO is fed in in process step f) at a CO partial pressure in the range of 1 to 5 MPa (10 to 40 bar).

In one variant of the process, the reaction mixture is heated in process step g) to a temperature in the range from 40° C. to 140° C.

In one variant of the process, the reaction mixture is heated in process step g) to a temperature in the range from 80° C. to 140° C.

In one variant of the process, the ethylenically unsaturated compound used is diisobutene.

In one variant of the process, the ethylenically unsaturated compound used is a mixture of 2,4,4-trimethylpent-1-ene (TMP1) and 2,4,4-trimethylpent-2-ene (TMP2).

In one variant of the process, the co-catalyst is aluminium triflate.

The invention shall be elucidated in more detail hereinbelow with reference to working examples.

### GENERAL PROCEDURES

The process is carried out under an argon atmosphere. Reaction vessels have been dried beforehand at high temperature (80° C.) and under oil-pump vacuum. Liquid substances are degassed by bubbling in argon for at least 15 minutes. The acid used is aluminium trifluoromethanesulfonic acid (Al(OTf)₃). The ligands used are (1), (2), (3) and the comparative ligand used is (4). The precursor used is palladium(II) bis(acetylacetonate) (Pd(acac)₂). The substrate used is diisobutene as a mixture of the two C8 isomers 2,4,4-trimethylpent-1-ene (TMP1) and 2,4,4-trimethylpent-2-ene (TMP2) in a ratio of 79:21.

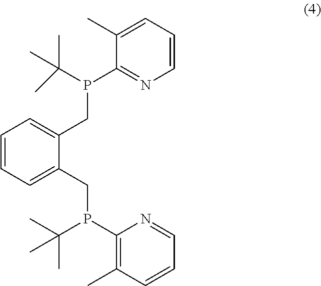 [SMILES: Cc1cccnc1P(Cc1ccccc1CP(c1ncccc1C)C(C)(C)C)C(C)(C)C]

### Conversion of the Substrate Diisobutene (DiB)

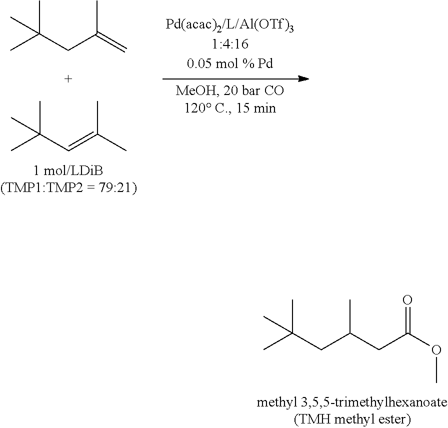

### Precursor Stock Solution:

Pd(acac)₂ (10 mg, 33 μmol) is weighed into a 20 mL vial, sealed in an airtight manner by means of a flanged septum, and dissolved in methanol (10 mL).

The reaction is conducted in 20 mL glass vessels with magnetic stirrer bars. Firstly, Al(OTf)₃ (0.8 mol %) and the ligand (0.2 mol %) are weighed into the glass vessel and then sealed in an airtight manner by means of a flanged septum. By means of a cannula pierced through, which is connected to an argon distribution station, an argon atmosphere is guaranteed in the steps which follow and there is at the same time the possibility of pressure equalization (addition of solutions). The amount of precursor stock solution required (1.5 ml) is added by means of a μL syringe, so as to result in a starting weight of Pd(acac)₂ (0.05 mol %). Finally, methanol is added by means of a μL syringe, so that a total volume of 8.4 mL is present. The autoclave is sealed, purged three times with nitrogen and the seal tested with 20 bar nitrogen. After establishing the seal, the same procedure is carried out with CO. The reaction solutions are then heated up to the required temperature of 120° C. After 20 minutes at constant temperature, the substrate is transferred into the reaction vessels by means of an HPLC pump. After 15 minutes, a sample is taken via each substrate line.

The conversions are listed in the following table:

| Ligand | Conversion (TMP1) [%] | Conversion (TMP2) [%] |
|---|---|---|
| (1)* | 55 | 14 |
| (2)* | 74 | 25 |
| (3)* | 62 | 23 |
| (4) | 0 | 0 |

* inventive working example

The experiments carried out demonstrate that the stated object is achieved by a compound according to the invention.

1. Compound of formula (I): 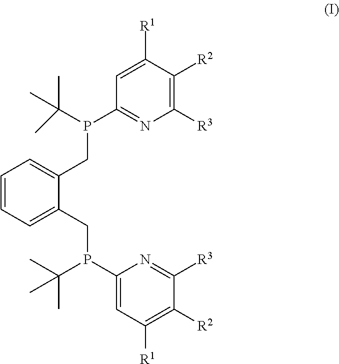
wherein one of the three radicals R¹, R², R³ is —(C₁-C₄)-alkyl, and the other two radicals are —H.

2. Compound according to claim 1,
wherein one of the three radicals R¹, R², R³ is —CH₃, and the other two radicals are —H.

3. Compound according to claim 1, wherein the compound has the structure (1):

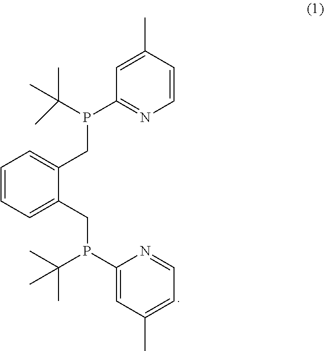 [SMILES: Cc1ccnc(P(Cc2ccccc2CP(c2cc(C)ccn2)C(C)(C)C)C(C)(C)C)c1]

4. Compound according to claim 1, wherein the compound has the structure (2):

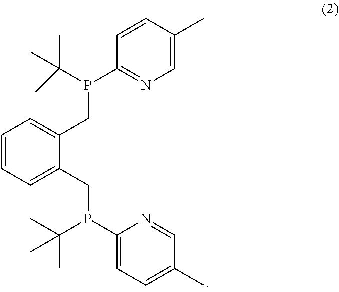 [SMILES: Cc1ccc(P(Cc2ccccc2CP(c2ccc(C)cn2)C(C)(C)C)C(C)(C)C)nc1]

5. Compound according to claim 1, wherein the compound has the structure (3):

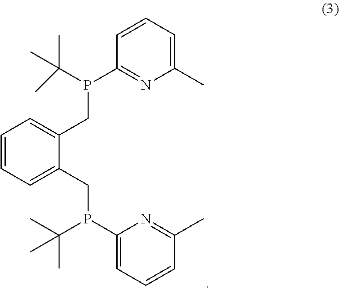 [SMILES: Cc1cccc(P(Cc2ccccc2CP(c2cccc(C)n2)C(C)(C)C)C(C)(C)C)n1]

6. Process comprising the process steps of:
a) initially charging an ethylenically unsaturated compound;
b) adding a compound according to claim 1;
c) adding a Pd compound;
d) adding a co-catalyst selected from: aluminium triflate, H₂SO₄, MSA, pTSA, TFA;
e) adding an alcohol;
f) feeding in CO;
g) heating the reaction mixture from a) to f), with conversion of the ethylenically unsaturated compound to an ester.

7. Process according to claim 6, wherein the Pd compound is selected from: palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl) dichloride.

8. Process according to claim 6, wherein the alcohol in process step e) is selected from: methanol, ethanol, 1-propanol, 1-butanol, 1-pentanol, 1-hexanol, 2-propanol, tert-butanol, 3-pentanol, cyclohexanol, phenol, or mixtures thereof.

9. Process according to claim 6, wherein the alcohol in process step e) is methanol.

10. Process according to claim 6, wherein CO is fed in in process step f) at a CO partial pressure in the range of 1 to 5 MPa (10 to 50 bar).

11. Process according to claim 6, wherein the reaction mixture is heated in process step g) to a temperature in the range from 40° C. to 140° C.

12. Process according to claim 6, wherein the ethylenically unsaturated compound used is diisobutene.

13. Process according to claim 6, wherein the co-catalyst is aluminium triflate.
